(19) United States
(12) Patent Application Publication
(10) Pub. No.: US 2025/0134753 A1
(43) Pub. Date: May 1, 2025

(54) NIPPLE CARE DEVICE PAD, AND HANDS-FREE INTEGRAL TYPE ELECTROMOTIVE NIPPLE CORRECTOR TO WHICH NIPPLE CARE DEVICE PAD IS APPLIED

(71) Applicant: NOVON CO.,LTD, Cheonan-si (KR)

(72) Inventor: Sangha KIM, Cheonan-si (KR)

(73) Assignee: NOVON CO.,LTD, Cheonan-si (KR)

(21) Appl. No.: 18/690,178

(22) PCT Filed: Oct. 25, 2022
(86) PCT No.: PCT/KR2022/016313

§ 371 Date: Mar. 7, 2024

(30) Foreign Application Priority Data
Oct. 29, 2021 (KR) 10-2021-0146394

(51) Int. Cl. A61H 9/00; A61H 19/00; A61M 1/00

(52) U.S. Cl. CPC A61H 9/0057; A61H 19/32; A61M 1/79; A61H 2201/1207; A61H 2201/1619; A61H 2201/165; A61H 2205/082; A61M 2210/1007

(57) ABSTRACT

Disclosed is a nipple care device pad.

The nipple care device pad of the present invention includes a close contact part having a predetermined area, a nipple loading part protruding in one direction of the close contact part and having a predetermined space therein so that a nipple is positioned in the space, a nipple pressure application hole formed at an end of the nipple loading part so that a pressure is applied to the nipple, and a filter part selectively seated in the nipple loading part and configured to filter out secretions released from the nipple to which the pressure is applied.

### TECHNICAL FIELD

The present invention relates to a nipple care device pad and a hands-free integral type electromotive nipple corrector to which the nipple care device pad is applied.

### BACKGROUND ART

In general, an inverted nipple refers to a condition in which the nipple is recessed from a surface of a breast because of imbalance in development of a milk-producing glands and milk ducts. It is estimated that about 7% of Korean women have inverted nipples.

The inverted nipple is not only unsightly but also causes secretions to accumulate in the inverted nipple, which is prone to offensive odor and inflammation.

In particular, after childbirth, when the breasts swell because of the hormonal effects, the nipples become more recessed into the breast, which causes great difficulty in feeding.

In the related art, this problem has been solved by massaging the breasts with frequent hand stimulation or using a milking machine to correct the breasts. However, these methods are not only cumbersome but also ineffective.

In addition, there is also a method of correcting inverted nipples through plastic surgery. However, this method has the practical problem that it is difficult to correct through plastic surgery after childbirth.

Therefore, in some instances, manual nipple correctors are used to correct the nipples.

However, the nipple corrector in the related art requires the user to manually press a manual pump to correct the nipple, which causes a lack of convenience of use and does not provide a sufficient correction function.

In addition, the nipple corrector does not provide the customized correction that each user needs, which makes it difficult for users to receive the appropriate nipple correction.

### DISCLOSURE

### Technical Problem

An object of the present invention is to provide a nipple care device pad capable of preventing contamination and an erroneous operation of a device by filtering out secretions released from a nipple during a correction process performed by the device for correcting an inverted nipple, and a hands-free integral type electromotive nipple corrector to which the nipple care device pad is applied.

### Technical Solution

Disclosed is a nipple care device pad.

The nipple care device pad of the present invention includes a close contact part having a predetermined area, a nipple loading part protruding in one direction of the close contact part and having a predetermined space therein so that a nipple is positioned in the space, a nipple pressure application hole formed at an end of the nipple loading part so that a pressure is applied to the nipple, and a filter part selectively seated in the nipple loading part and configured to filter out secretions released from the nipple to which the pressure is applied.

### Advantageous Effects

According to the present invention configured as described above, it is possible to prevent contamination and an erroneous operation of the device by filtering out secretions released from the nipple during the process of correcting an inverted nipple.

### DESCRIPTION OF DRAWINGS

FIGS. 1 and 2 are perspective views illustrating front and rear sides of a nipple care device pad of the present invention.

FIG. 3 is a coupled perspective view of a filter part that is one component of the present invention.

FIG. 4 is a view illustrating a fastened state of the filter part.

FIGS. 5 to 9 are views illustrating a hands-free integral type electromotive nipple corrector to which the nipple care device pad is fastened.

FIG. 10 is a view for explaining a stepped portion that is one component of the present invention.

FIG. 11 is an actual photograph for explaining a pressure supply hole that is one component of the present invention.

FIGS. 12 to 14 are views related to previously registered Korean Patent No. 10-1928800.

FIGS. 15 to 18 are views illustrating experimental results related to water permeability experiments on a filter of the present invention.

### MODES OF THE INVENTION

Hereinafter, exemplary embodiments of a nipple care device pad and a hands-free integral type electromotive nipple corrector to which the nipple care device pad is applied according to the present invention will be described with reference to the accompanying drawings.

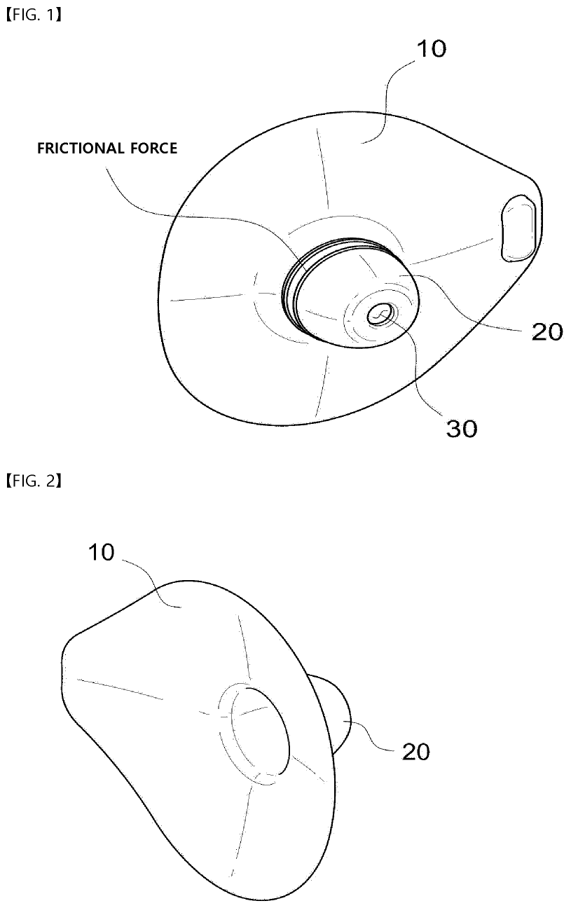

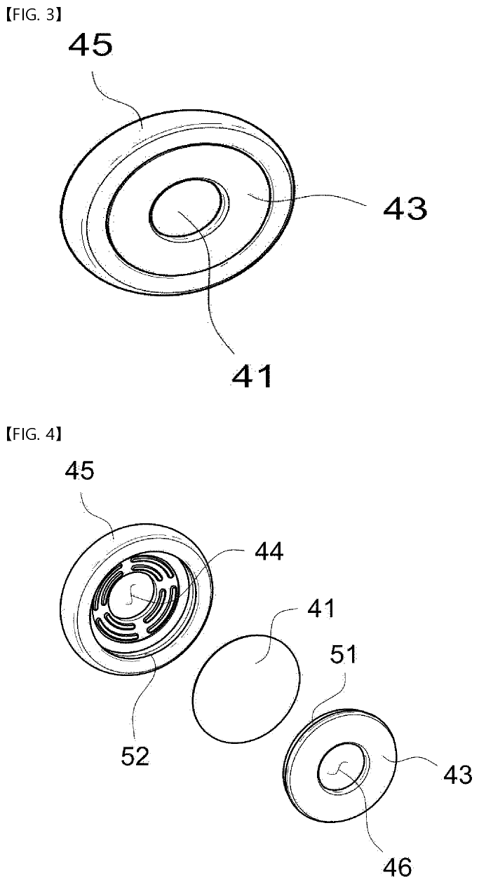

FIGS. 1 and 2 are perspective views illustrating front and rear sides of a nipple care device pad of the present invention, FIG. 3 is a coupled perspective view of a filter part 40 that is one component of the present invention, and FIG. 4 is a view illustrating a fastened state of the filter part 40.

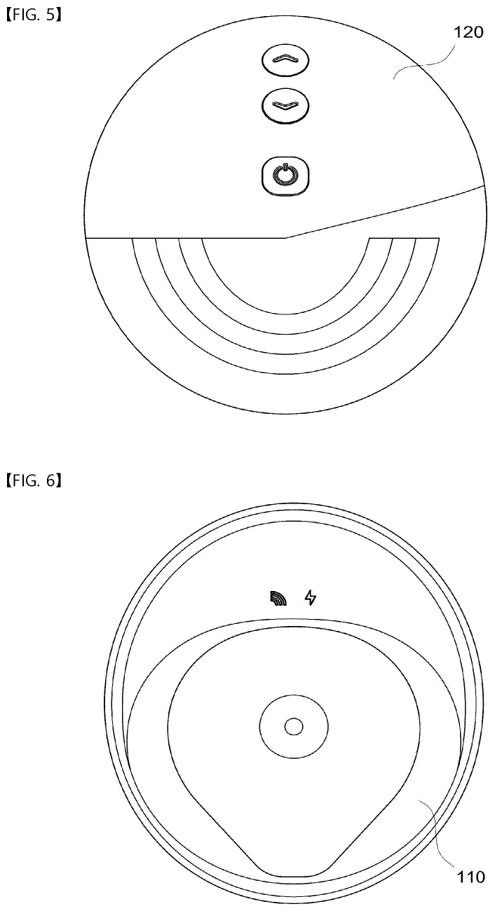

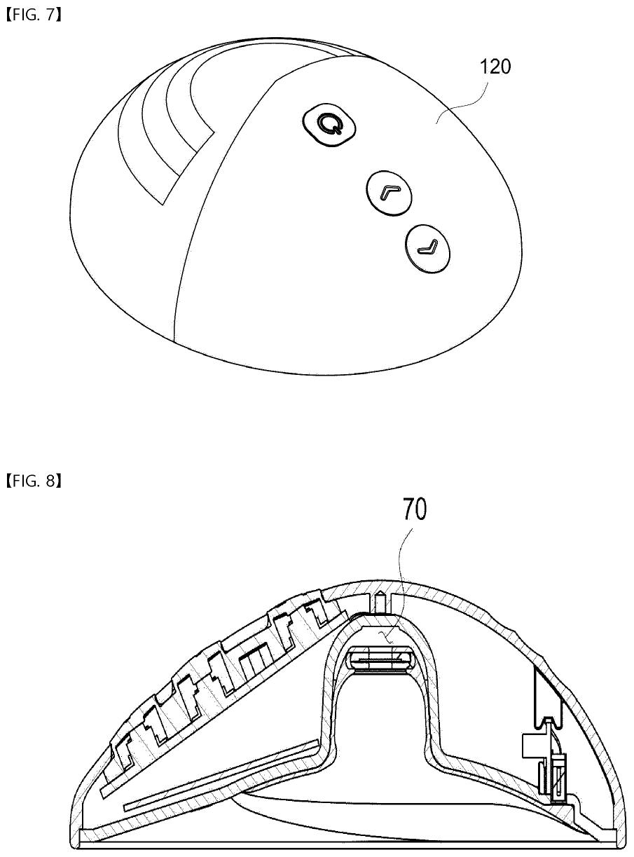

FIGS. 5 to 9 are views illustrating a hands-free integral type electromotive nipple corrector to which the nipple care device pad is fastened.

As illustrated, the present invention provides a close contact part 10 having a predetermined area.

The close contact part 10 has, but not necessarily limited to, a circular shape as a whole. The close contact part 10 is positioned while being in close contact with a suction part 110 illustrated in FIG. 6.

Meanwhile, a nipple loading part 20 protrudes in one direction of the close contact part 10 and has a predetermined space therein, and the nipple may be positioned in the space.

As described above, the nipple is positioned in the nipple loading part 20, and a pressure to be described below is applied.

A nipple pressure application hole 30 is formed at an end of the nipple loading part 20 so that a pressure may be applied to the nipple.

A pressure, which is applied by a pump for applying a suction pressure to be described below, is transmitted to the nipple through the nipple pressure application hole 30.

Meanwhile, the filter part 40, which is a key technical feature of the present invention, is illustrated in FIGS. 3 and 4.

The filter part 40 is selectively seated in the nipple loading part 20 and filters out secretions released from the nipple to which the pressure is applied.

The inverted nipple is corrected by the pressure applied to the nipple, but at the same time, secretions are released from the nipple by the pressure.

The secretions cause offensive odors and contamination, and the secretions cause a defect or erroneous operation of an electronic device. The present invention is intended to solve this problem by using the filter part 40.

Meanwhile, the filter part 40, which is an essential component of the present invention, will be described with reference to FIGS. 3 and 4.

As illustrated, the filter part 40 has a filter 41 made of a membrane material, and the filter 41 is fastened to a filter fixing member 45 having a second hole 44 formed at a center thereof.

A filter seating member 43 having a first hole 46 corresponding to the second hole 44 is fastened to the filter fixing member 45 to prevent the filter 41 from separating from the filter fixing member 45.

A pressure is applied along the first hole 46 and the second hole 44, and the pressure passing through the filter 41 corrects the inverted nipple.

A Versapor membrane filter 41 may be used as the membrane filter 41, but the present invention is not necessarily limited thereto.

Meanwhile, a plurality of first screw threads 51 is formed on an outer peripheral surface of the filter seating member 43, and second screw threads 52, which may correspond to and engage with the first screw threads 51, are formed on an inner peripheral surface of the filter fixing member 45.

In this case, as described above, the coupling relationship may be implemented by the engagement between the screw threads, but the present invention is not necessarily limited thereto. The filter seating member 43 and the filter fixing member 45 may be fastened in an interference-fit manner, and the present invention may be implemented by ultrasonic welding.

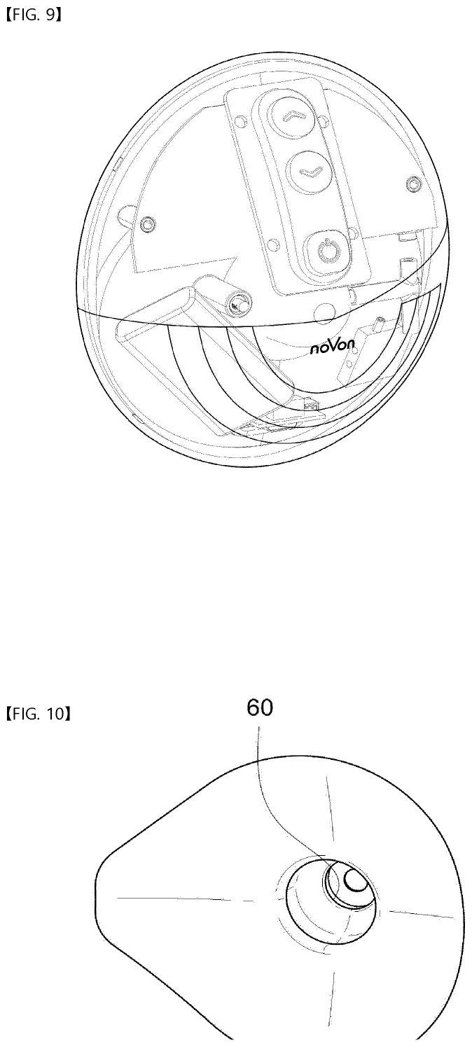

FIG. 10 is a view for explaining a stepped portion 60 that is one component of the present invention.

As illustrated, the stepped portion 60 is formed at one point in the nipple loading part 20 and formed along a circumference of an inner peripheral surface of the nipple loading part 20. The filter part 40 is selectively fitted with the stepped portion 60.

The stepped portion 60 allows the filter part 40 to be stably fastened.

Hereinafter, the hands-free integral type electromotive nipple corrector to which the nipple care device pad is applied will be described.

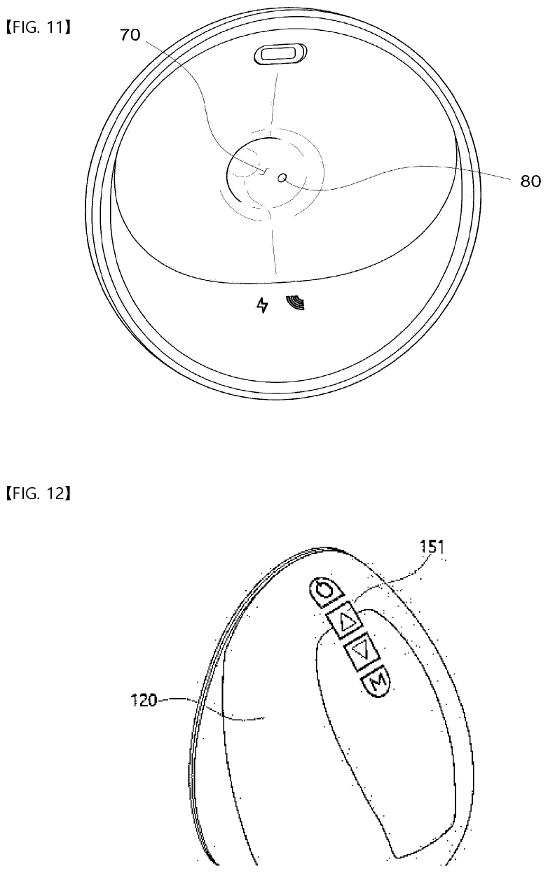

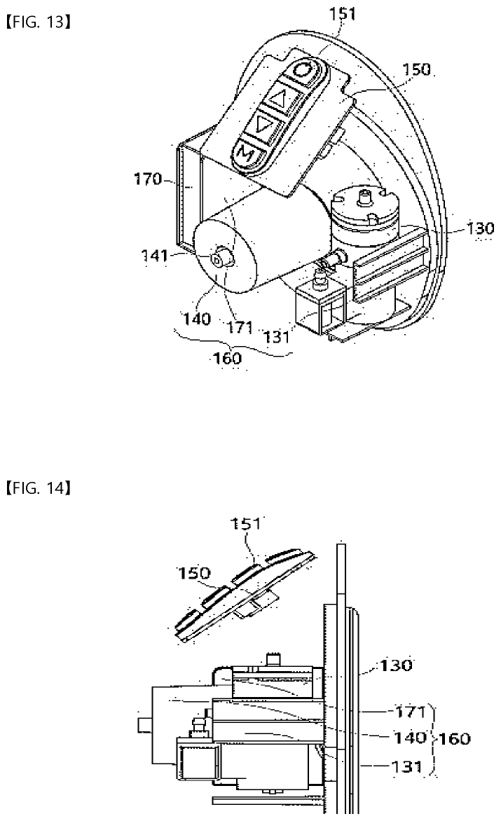

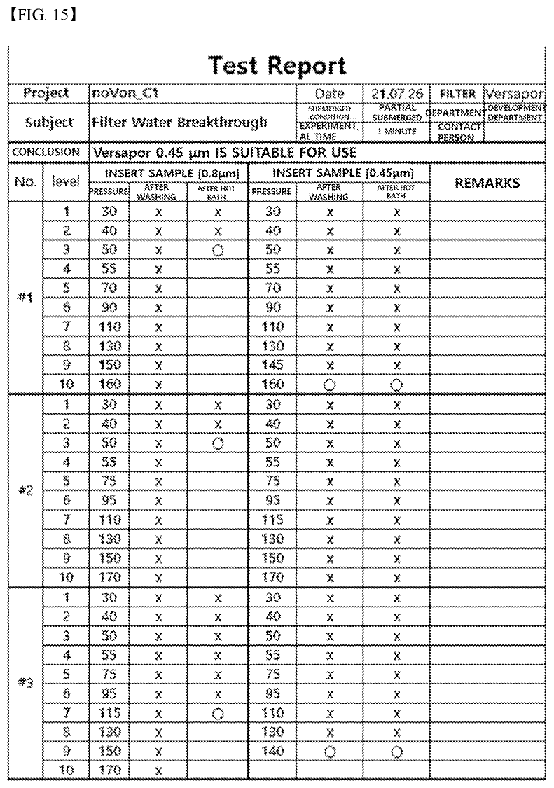

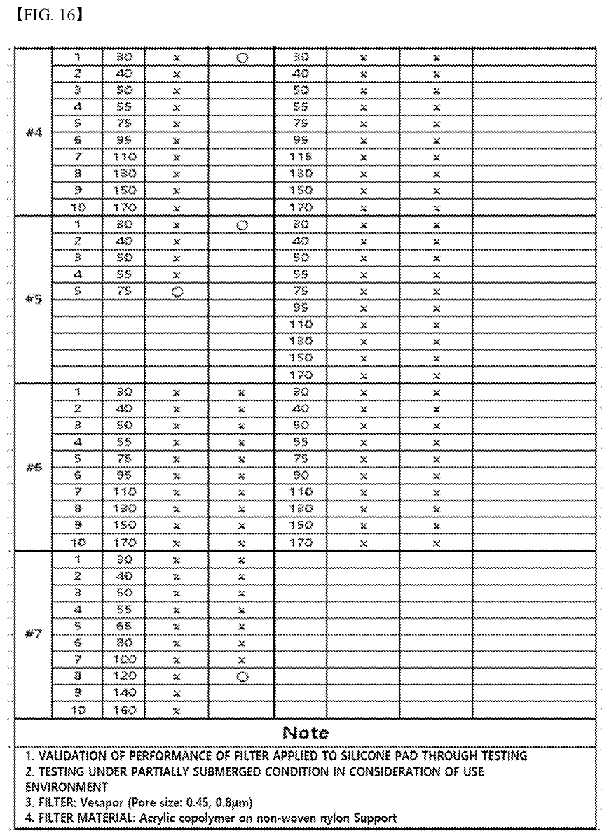

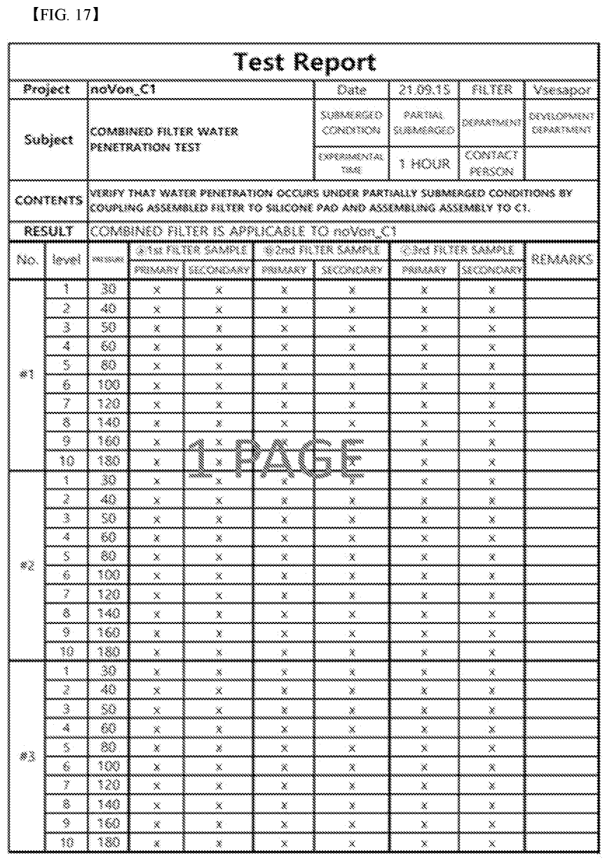

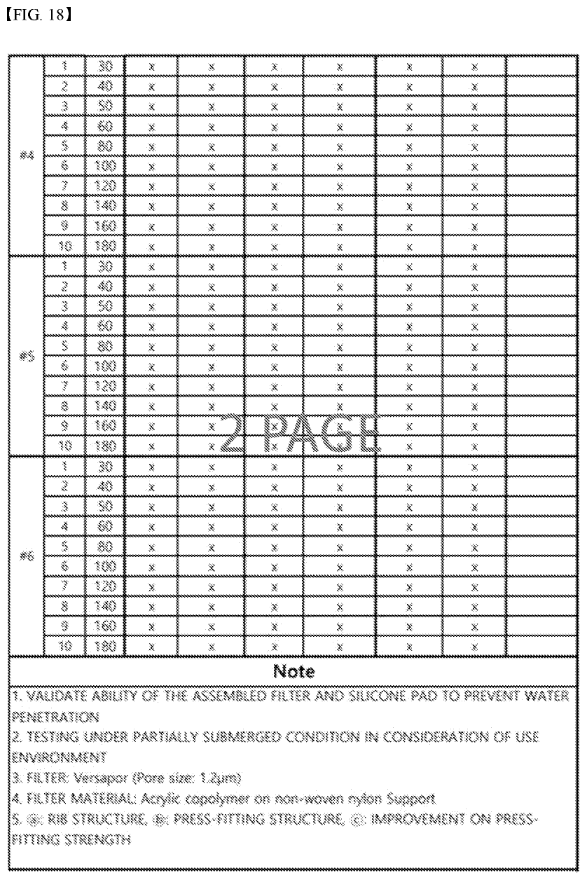

FIG. 11 is an actual photograph for explaining a pressure supply hole that is one component of the present invention, and FIGS. 12 to 14 are views related to previously registered Korean Patent No. 10-1928800.

The present invention relates to the hands-free integral type electromotive nipple corrector to which the nipple care device pad is applied. The hands-free integral type electromotive nipple corrector has the suction part 110 attached to a user's breast, and a pump configured to apply a suction pressure. The pump is connected to the suction part 110 and applies a suction pressure to the suction part 110. The hands-free integral type electromotive nipple corrector is formed in a cylindrical shape and opened at a side of the suction part 110.

At the other side opposite to the opened side, a pressure application part is provided and has a pump connection portion connected to the pump through an air hose. A controller is provided to control an operation of the pump in response to a control instruction. An inlet 70 having a predetermined depth is formed in the suction part 110.

A pressure supply hole 80 is formed at one side of an inner peripheral surface of the inlet 70 so that the pressure may be applied by the pump.

In this case, as illustrated in FIG. 11, the pressure supply hole 80, to which the pressure from the pump may be applied, is formed at one side of the inner peripheral surface of the inlet 70, and the pressure is transmitted by the pressure supply hole 80.

In addition, as described above, the close contact part 10 is in close contact with one side surface of the suction part 110 and has a predetermined area. The nipple loading part protrudes in one direction of the close contact part 10 and has a predetermined space therein.

The nipple is positioned in the space. The nipple loading part 20 is introduced into the inlet 70. The nipple pressure application hole 30 is formed at the end of the nipple loading part 20 to apply the pressure to the nipple. The filter part 40 is selectively seated in the nipple loading part 20 and filters out secretions released from the nipple to which the pressure is applied.

Because the above-mentioned structure has been described above, a description thereof will be omitted.

As illustrated, the hands-free integral type electromotive nipple corrector according to the embodiment of the present invention may include the suction part 110, a cover part 120, a pump 130, a pressure application part 140, and a controller 150.

The suction part 110 and the cover part 120 may be coupled to constitute an external appearance. An instruction input part 151 may be provided on the cover part 120 and provided in the form of a button in order to control the operations of operating or stopping the nipple corrector and adjusting intensity.

The suction part 110 is attached to be in close contact with the user's breast, and the pump 130 applies the suction pressure to the suction part 110, such that the user's nipple is corrected by the suction pressure.

The close contact part 10 is in close contact with the suction part 110, as described above.

The pressure application part 140 is opened toward the suction part 110 to apply the suction pressure to the suction part 110. A pump connection portion 141 may be formed at the other side of the pressure application part 140 and connected to the pump 130 through the air hose.

Of course, in the present invention, there is no opened portion, and the close contact part 10 is seated on the corresponding portion.

Therefore, the pressure, which is applied to the pressure application part 140 by the pump 130, reduces the pressure in the space in the suction part 110 attached to the user's breast, such that the user's nipple may be sucked and corrected.

In the present invention, the pressure from the pressure application part is applied along the pressure supply hole 80 and transmitted along the nipple pressure application hole 30, and the pressure is applied along the first hole 46 and the second hole 44 of the filter part 40 and then transmitted to the nipple.

The controller 150 may control the operations of operating or stopping the nipple corrector and adjusting the intensity by controlling the operation of the pump 130 in response to a control instruction.

The instruction input part 151 for inputting the control instruction for the nipple corrector may be disposed to be exposed through the cover part 120.

It is possible to provide the user with a variety of personalized correction options, which allows the user to select the appropriate correction based on the user's individual needs.

Meanwhile, the cover part 120 may have a dome-shaped external appearance to allow the user to easily grip the cover part 120.

To facilitate the user's manipulation, the instruction input part 151 may be disposed at the upper side of the cover part 120 corresponding to a portion where the user's index finger is positioned when the user grips the cover part 120.

In addition, to improve portability and convenience of use for the user, the hands-free integral type electromotive nipple corrector according to the embodiment of the present invention may not be supplied with power from the outside, but may include a battery 170, such that the battery 170 may supply power for operating the pump 130.

The pressure application part 140 is formed in a cylindrical shape and opened at a side directed toward the suction part 110. The pump connection portion 141 may be formed at the other side opposite to the opened side and connected to the pump 130 through the air hose.

Therefore, with the above-mentioned structure of the pressure application part 140, the pressure applied from the pump 130 may be more effectively transmitted to the suction part 110 through the pressure application part 140.

Furthermore, in the hands-free integral type electromotive nipple corrector according to the embodiment of the present invention, a coupling part 160, which is coupled to the suction part 110, may have an integral type structure.

More specifically, the coupling part 160 may have the integral type structure including a pump support part 131 configured to support the pump 130, the pressure application part 140, and a battery accommodation part 171 configured to accommodate the battery 170.

Therefore, the above-mentioned structure, in which the coupling part 160 and the cover part 120 are coupled, may constitute the external appearance of the hands-free integral type electromotive nipple corrector and configure the compact structure for accommodating the internal components of the hands-free integral type electromotive nipple corrector.

1. A nipple care device pad comprising:
a close contact part (10) having a predetermined area;
a nipple loading part (20) protruding in one direction of the close contact part (10) and having a predetermined space therein so that a nipple is positioned in the space;
a nipple pressure application hole (30) formed at an end of the nipple loading part (20) so that a pressure is applied to the nipple; and
a filter part (40) selectively seated in the nipple loading part (20) and configured to filter out secretions released from the nipple to which the pressure is applied.

2. The nipple care device pad of claim 1, wherein the filter part (40) has a filter (41) made of a membrane material, and the filter (41) is fastened to a filter fixing member (45) having a second hole (44) formed at a center thereof, and a filter seating member (43) having a first hole (46) corresponding to the second hole (44) is fastened to the filter fixing member (45) to prevent the filter (41) from separating from the filter fixing member (45).

3. The nipple care device pad of claim 2, wherein a plurality of first screw threads (51) is formed on an outer peripheral surface of the filter seating member (43), and second screw threads (52), which correspond to and engage with the first screw threads (51), are formed on an inner peripheral surface of the filter fixing member (45).

4. The nipple care device pad of claim 2, wherein the filter seating member (43) and the filter fixing member (45) are fastened in an interference-fit manner.

5. The nipple care device pad of claim 2, wherein the filter seating member (43) and the filter fixing member (45) are coupled by ultrasonic welding.

6. The nipple care device pad of claim 3, wherein a stepped portion (60) is formed at one point in the nipple loading part (20) and formed along a circumference of an inner peripheral surface of the nipple loading part (20), and the filter part (40) is selectively fitted with the stepped portion (60).

7. A hands-free integral type electromotive nipple corrector to which a nipple care device pad is applied, the hands-free integral type electromotive nipple corrector comprising:
a suction part (110) configured to be attached to a user's breast;
a pump configured to apply a suction pressure;
a pressure application part connected to the suction part (110) to apply the suction pressure from the pump to the suction part (110), having a cylindrical shape, and opened at a side directed toward the suction part (110), wherein a pump connection portion, which is connected to the pump through an air hose, is formed at the other side opposite to the opened side; and
a controller configured to control an operation of the pump in response to a control instruction,
wherein an inlet (70) having a predetermined depth is formed in the suction part (110),
wherein a pressure supply hole (80) is formed at one side of an inner peripheral surface of the inlet (70) so that a pressure from the pump is applied, and
wherein the nipple care device pad comprises:
a close contact part (10) configured to be in close contact with one side surface of the suction part (110) and having a predetermined area;
a nipple loading part (20) protruding in one direction of the close contact part (10) and having a predetermined space therein so that a nipple is positioned in the space, the nipple loading part (20) being introduced into the inlet (70);
a nipple pressure application hole (30) formed at an end of the nipple loading part (20) so that a pressure is applied to the nipple; and
a filter part (40) selectively seated in the nipple loading part (20) and configured to filter out secretions released from the nipple to which the pressure is applied.

8. The hands-free integral type electromotive nipple corrector of claim 7, wherein the filter part (40) has a filter (41) made of a membrane material, and the filter (41) is fastened to a filter fixing member (45) having a second hole (44) formed at a center thereof, and a filter seating member (43) having a first hole (46) corresponding to the second hole (44) is fastened to the filter fixing member (45) to prevent the filter (41) from separating from the filter fixing member (45).

9. The hands-free integral type electromotive nipple corrector of claim 8, wherein a plurality of first screw threads (51) is formed on an outer peripheral surface of the filter seating member (43), and second screw threads (52), which correspond to and engage with the first screw threads (51), are formed on an inner peripheral surface of the filter fixing member (45).

10. The hands-free integral type electromotive nipple corrector of claim 8, wherein the filter seating member (43) and the filter fixing member (45) are fastened in an interference-fit manner.

11. The hands-free integral type electromotive nipple corrector of claim 8, wherein the filter seating member (43) and the filter fixing member (45) are coupled by ultrasonic welding.

12. The hands-free integral type electromotive nipple corrector of claim 9, wherein a stepped portion (60) is formed at one point in the nipple loading part (20) and formed along a circumference of an inner peripheral surface of the nipple loading part (20), and the filter part (40) is selectively fitted with the stepped portion (60).

13. The hands-free integral type electromotive nipple corrector of claim 10, wherein a stepped portion (60) is formed at one point in the nipple loading part (20) and formed along a circumference of an inner peripheral surface of the nipple loading part (20), and the filter part (40) is selectively fitted with the stepped portion (60).

14. The nipple care device pad of claim 4, wherein a stepped portion (60) is formed at one point in the nipple loading part (20) and formed along a circumference of an inner peripheral surface of the nipple loading part (20), and the filter part (40) is selectively fitted with the stepped portion (60).

15. The nipple care device pad of claim 5, wherein a stepped portion (60) is formed at one point in the nipple loading part (20) and formed along a circumference of an inner peripheral surface of the nipple loading part (20), and the filter part (40) is selectively fitted with the stepped portion (60).
